(19) United States
(12) Patent Application Publication
(10) Pub. No.: US 2020/0219624 A1
(43) Pub. Date: Jul. 9, 2020

(54) METHOD FOR MAINTAINING A HEALTHY MASS

(71) Applicant: Erlan H. Feria, Princeton, NJ (US)

(72) Inventor: Erlan H. Feria, Princeton, NJ (US)

(21) Appl. No.: 16/820,121

(22) Filed: Mar. 16, 2020

Related U.S. Application Data
(63) Continuation-in-part of application No. 14/875,064, filed on Oct. 5, 2015.
Application No. 14/875,064 is a continuation-in-part of application No. 14/243,149, filed on Apr. 2, 2014, now abandoned.
Application No. 14/243,149 is a continuation-in-part of application No. 13/646,224, filed on Oct. 5, 2012, now abandoned.
(60) Provisional application No. 62/059,273, filed on Oct. 3, 2014.
(60) Provisional application No. 61/807,363, filed on Apr. 2, 2013.
(60) Provisional application No. 61/544,838, filed on Oct. 7, 2011.

(51) Int. Cl. G16H 50/30; G16H 20/30; G16H 20/60; A61B 5/00; G01G 19/44

(52) U.S. Cl. CPC G16H 50/30; G16H 20/30; G01G 19/44; A61B 5/4866; G16H 20/60

(57) ABSTRACT

The subject matter disclosed pertains to a method of maintaining a healthy mass. A target nutritional consumption rate (ΔM) is calculated using linger thermodynamic theory. Both diet and exercise are adjusted to accommodate this target nutritional consumption rate (ΔM). The disclosed method minimizes metabolic strain and thereby maximizes life expectancy.

### CROSS-REFERENCE TO RELATED APPLICATIONS

This application claims priority to and is a continuation-in-part of U.S. patent application Ser. No. 14/875,064 (filed Oct. 5, 2015) which claims the benefit of U.S. provisional patent application 62/059,273 (filed Oct. 3, 2014) and is a continuation-in-part of U.S. patent application Ser. No. 14/243,149 (filed Apr. 2, 2014) which claims priority to U.S. non-provisional patent application 61/807,363 (filed Apr. 2, 2013). U.S. patent application Ser. No. 14/243,149 is also a continuation-in-part of U.S. application Ser. No. 13/646,224 (filed Oct. 5, 2012) which claims priority to and the benefit of U.S. provisional patent application 61/544,838 (filed Oct. 7, 2011). All of the above-mentioned patent applications are incorporated herein by reference in their entirety.

### BACKGROUND OF THE INVENTION

Life expectancy can be predicted based on a variety of parameters including the individual's demographic data and medical history including weight. Traditionally, insurance companies utilize actuarial tables and other calculations in an attempt to predict the individual's life expectancy. This predicted life expectancy, in turn, impacts the individual's life insurance premium. Those individuals with a short life expectancy pay high premiums while those with relatively long life expectancy pay lower premiums.

Unfortunately, the actuarial tables only correlate some variables which are currently believed to impact life expectancy. Additional medical studies have discovered new variables that the current tables fail to consider. It would be desirable to provide an improved method for calculating life expectancy that takes into account additional variables so as to provide more accurate life expectancy predictions.

The discussion above is merely provided for general background information and is not intended to be used as an aid in determining the scope of the claimed subject matter.

### BRIEF DESCRIPTION OF THE INVENTION

The subject matter disclosed pertains to a method of maintaining a healthy weight. The level of caloric intake and expenditure is determined according to universal linger thermodynamic theory.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a graph of the mathematical relationship between body mass and nutritional consumption rate and the resulting impact on adult lifespan;

FIG. 2 is a flow diagram of an exemplary method for maintaining a desired mass; and

FIG. 3 is a graph of the mathematical relationship between specific heat and the resulting impact on adult lifespan;

FIG. 4 is an exemplary computer system for executing the operations of an application program for carrying out the calculation of life expectancy in accordance with this disclosure.

### DETAILED DESCRIPTION OF THE INVENTION

The subject matter disclosed herein relates to the calculation of desirable caloric consumption and expenditure to maintain a healthy weight. Traditional calculations and actuarial tables often presume that individuals with a high weight are unhealthy. For example, in a recent study, researchers were surprised to discover that low-weight rhesus monkeys had the same life expectancy as higher weight monkeys (Kolata, Severe Diet doesn't Prolong Life, at Least in Monkeys, The New York Times, Aug. 29, 2012).

In one embodiment, the life expectancy calculations described herein considers the ratio of the individual's mass to their nutritional consumption rate. High-mass individuals consume food at an appropriately high nutritional consumption rate. Likewise, low-mass individuals consume food at a correspondingly reduced nutritional consumption rate. As shown by the rhesus monkey studies, these low-mass individuals are not more likely to have a longer life expectancy simply due to their lower mass. Without wishing to be bound to any particular theory, the ratio of the mass to the nutritional consumption rate provides a quantitative measure of the stresses experienced by the individual person's body. For example, if two individuals have equal mass (e.g. both 70 kg) the individual who consumes more energy (while maintaining weight) is experiencing more metabolic strain. This results in reduced life expectancy despite the controlled weight. The disclosed method has been developed to address this shortcoming.

In another embodiment, the life expectancy calculations described herein consider the specific heat capacity (Cᵥ) of an individual. Without wishing to be bound to any particular theory, the specific heat capacity (Cᵥ) provides a quantitative measure of the stresses experienced by the individual person's body. For example, if two individuals have equal mass (e.g. both 70 kg) the individual with the higher specific heat capacity (Cᵥ) (while maintaining their weight) is experiencing more metabolic strain. This results in reduced life expectancy despite the controlled weight.

### Nutritional Consumption Rate Embodiment

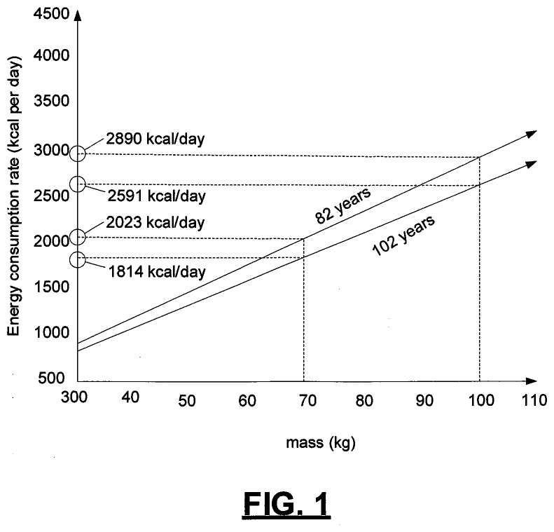

FIG. 1 is a graph showing a mathematical relationship between nutritional consumption rate (ΔM) and mass (M) of a person according to the following equation:

$${τ}_{t h e o r {y}_{-} a dult} = Δ {τ ( \frac{M}{Δ M} )}^{2} \qquad equation 1$$

In FIG. 1, an upper line shows 82 years of a theoretical adult lifespan (τ_{theory_adult}) while a lower line shows 102 years of a theoretical adult lifespan. An individual person who weighs 70 kg intercepts the 102-year-line when approximately 1814 kcal per day are consumed. Another individual with the same 70 kg mass is predicted to have a theoretical adult lifespan of 82 years if 2023 kcal per day are consumed. In a similar fashion, an individual person who weighs 100 kg is predicted to have a theoretical adult lifespan of 102 years when 2591 kcal per day are consumed but an 82 year theoretical adult lifespan when 2890 kcal per day are consumed. The mathematical model disclosed herein accounts for the fact that a 100 kg individual consuming 2591 kcal per day can have a longer adult lifespan than a 70 kg individual eating 2023 kcal per day. Such information is useful in the creation of dietary and exercise plans for individuals.

Equation 1 can be rearranged to solve for a desired nutritional consumption rate (ΔM) for an individual to maintain a health mass (M).

$$Δ M = M \sqrt{\frac{Δ τ}{{τ}_{t h e o r {y}_{-} a d u l t}}} \qquad equation 2$$

In this equation Δτ is a conversion factor for converting between years and days (e.g. 1 year/365 days), M is the current mass a given individual wants to maintain and τ_{theory_adult} is the adult theoretical lifespan of 102 years. Given that 1 kg of food contains approximately 5000 kcal, one can solve for a target number of calories per day that should be both consumed and utilized.

### Example 1

By way of illustration, a 100 kg individual would solve the equation as follows:

$$\begin{matrix}Δ M = M \sqrt{\frac{Δ τ}{{τ}_{t h e o r {y}_{-} adult}}} = 100 kg \sqrt{\frac{1 year \text{/} 365 days}{102 years}} = 0.518 kg food & equation 3 \\ \frac{0.5 \; 18 kg food }{1} \frac{50000 kcal}{kg food} = 2591 kcal & equation 4\end{matrix}$$

The individual the follows a dietary plan that ensures they consume the calculated amount of food per day (e.g. 2591 kcal of food). The individual determines their basal metabolic rate (BMR) using conventional methods. For example, a 100 kg male who is 6′2″ and 48 years old may find his BMR to be 2,050 kcal per day. This individual would need to engage in an amount of exercise to burn an additional 541 kcal per day.

### Example 2

By way of illustration, a 70 kg individual would solve the equation as follows:

$$\begin{matrix}Δ M = M \sqrt{\frac{Δ τ}{{τ}_{{theory}_{-} adult}}} = 70 kg \sqrt{\frac{1 year \text{/} 365 days}{102 years}} = 0.363 kg food & equation 5 \\ \frac{0.3 \; 63 kg food}{1} \frac{50000 kcal}{kg food} = 1814 kcal \\ & equation 6\end{matrix}$$

The individual the follows a dietary plan that ensures they consume the calculated amount of food per day (e.g. 1814 kcal of food). The individual determines their basal metabolic rate (BMR) using conventional methods. For example, a 70 kg female who is 5′10″ and 35 years old may find her BMR to be 1,490 kcal per day. This individual would need to engage in an amount of exercise to burn an additional 324 kcal per day.

### Comparative Example

To illustrate a situation where metabolic stress reduces life expectancy, a second individual is used as an example. This individual is a 70 kg female who is 5′10″ and is 35 years old (BMR of 1,490 kcal per day). This particular individual consumes 3,814 kcal per day. To maintain the 70 kg weight, these individual exercises to burn an additional 2324 kcal per day (3814 kcal−1490 kcal). While this is adequate to maintain the 70 kg weight, this activity induces metabolic strain and actually decreases the individuals' life expectancy.

In one embodiment, a feedback loop is utilized to correct the daily food intake and the daily exercise amount for any errors in measurement. If the individual is gaining mass (e.g. as determined after a one week period), the individual reduces caloric intake (e.g. by 5% of calories) while maintaining the current level of exercise. Importantly, increasing exercise is not performed because this increases metabolic stress. Conversely, if the individual is losing mass, the individual reduces the daily amount of exercise (e.g. by 5% of the caloric burn) while maintaining the current level of food consumption. Importantly, decreasing food consumption is not performed because this increases metabolic stress. If the individual is neither gaining or losing mass, then the individual is successfully maintaining a healthy weight and continues with the current level of food consumption and exercise.

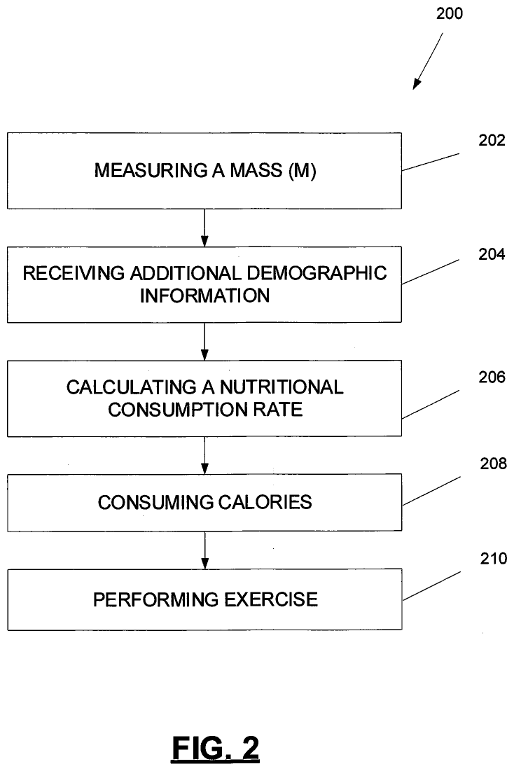

FIG. 2 is a flow diagram of method 200 for maintaining a desired mass. Method 200 begins with step 202 wherein a mass (M) for an individual person is received and inputted into a computer.

In step 204, one or more additional demographic parameters concerning the individual person are received and inputted into the computer. Examples of additional demographic parameters include height, age, waist circumference, hip circumference, gender, country of residency, diet, physique, exercise history, drug use (including tobacco and alcohol), personality disposition, level of education, ethnicity, medical history, family medical history, marital status, fitness, economic class, generalized body mass index (GBMI), body volume index (BVI), waist-to-hip-ratio (WHR), environmental/climate/geographic effects, sleep schedule, regularity of visits to healthcare providers and a quantified life-expectancy condition. The life-expectancy condition may be determined, for example, by actuarial tables. In one embodiment, a life-expectancy condition is a number greater than zero and equal to or less than one, with a value of one denoting an ideal condition. GBMI may be calculated by M/h^{c} where M is the individual's mass, h is their height, and c is a value that is set according to the demographics of the individual. C is often assigned values of 2, 2.3, 2.7 or 3 depending on the demographic.

In step 206 of method 200, a nutritional consumption rate (ΔM) is calculated according to Equation 2. The nutritional consumption rate is a quantitative measurement of the consumption and expenditure of nutrients over a given period of time. For example, mass of food consumed per day (e.g. kg per day) is one manner for expressing nutritional consumption rate. In another embodiment, the nutritional consumption rate is expressed in terms of energy per day (e.g. kcal per day). These two expressions can be inter-converted using a conversion factor (Y). For example, if one assumes that one kg of food supplies, on the average, 5000 kcal of energy, then one can convert a nutritional consumption rate of kcal per day into units of kg per day using a Y value of 5000 kcal per kg. The 5000 kcal per kg is merely one example. Other values of Y may also be used. An exemplary calculating using a 2000 kcal per day diet is shown below:

$$Δ M = \frac{X kcal}{day} \frac{kg}{Y kcal} = \frac{2000 kcal}{day} \frac{kg}{5000 kcal} = \frac{0.4 kg}{day} . \qquad equation 7$$

In step 208, a daily amount of food is consumed that is equal to the nutritional consumption rate as determined in step 206. This daily amount of food can be consumed pursuant to a conventional dietary plan. When operating in accordance with the disclosed method, consuming either more than or less than 1% of the calculated nutritional consumption rate (measured in kcal per day) is not recommended as this may induce metabolic stress and negatively impact life expectancy.

In step 210, the individual engages in exercise sufficient to bring his or her daily caloric expenditure equal to the nutritional consumption rate as determined in step 206. When operating in accordance with the disclosed method, engaging in either more than or less than 1% of an amount of caloric expenditure (measured in kcal per day) of the nutritional consumption rate is not recommended as this may induce metabolic stress and negatively impact life expectancy. The nutritional consumption rate (ΔM) shown above accounts for both the individual's mass (M) as well as the metabolic stress that results from exercise.

The theoretical total lifespan (Γ) includes both the childhood and adolescent years during which time the individual is still growing. In one embodiment, the childhood lifespan is set to a value of eighteen years. Depending on demographic and other variables, other values may be set for the childhood lifespan. A theoretical total lifespan (Γ) is determined according to:

Γ=τ_{theory_adult}+τ_{childhood} equation 8

The theoretical total lifespan comprises the theoretical adult lifespan. The theoretical adult lifespan and the theoretical total lifespan are both theoretical lifespans. An expected lifespan (F) is determined.

F=p_{A}(Γ−A) equation 9

The expected lifespan (F) is determined by subtracting the individual person's current age (A) from the theoretical total lifespan (Γ) and then adjusting for the probability of survival (p_{A}) from the age (A) to the theoretical total lifespan (Γ). The probability of survival (p_{A}) may be determined from actuarial tables that take other parameters into consideration. These parameters may be received, for example, in step 204 of method 200.

The maximum total lifespan (Γₘₐₓ) of human beings is not known with certainty but estimations of this value are often made. A maximum adult lifespan (τₘₐₓ) is set according to:

τₘₐₓ=Γₘₐₓ−τ_{childhood} equation 10

For example, some individuals believe the maximum total lifespan (Γₘₐₓ) is one-hundred twenty years. If one sets the childhood lifespan (τ_{childhood}) to eighteen years, then the maximum adult lifespan (τₘₐₓ) would be set to be equal to one-hundred two years. This value is one factor that is useful in determining the probability of survival (p_{A}) which is one of the factors in determining the expected lifespan (F).

In one embodiment, the probability of survival (p_{A}) is calculated according to the equation shown below, where P(x) is a positive number that is a function of the demographic parameter vector x where the value of P(x) is appropriately determined using actuarial tables.

$${p}_{A} = \frac{{τ}_{Max} + P ( x ) ( {τ}_{t h e o r {y}_{-} a d ult} - {τ}_{Max} )}{{τ}_{Max}} \qquad equation 11$$

### Specific Heat Embodiment

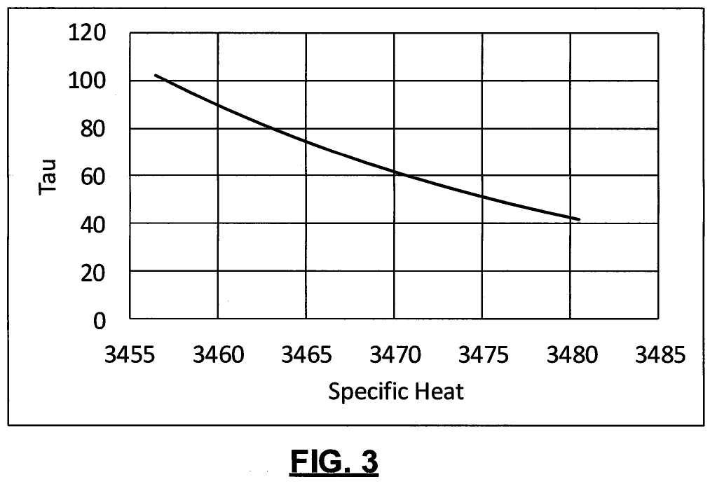

Referring to FIG. 3, in one embodiment, a theoretical adult lifespan (τ_{theory_adult}) calculated according to:

τ_{theory}=Δτ·3.515×10³¹(4.872×10⁻³⁸·Cᵥ^{specific})^{0.00048042(C}^{ᵥ}^{^{specific}}⁻¹⁷⁹⁴⁾ Equation 12:
where:
ΔT is a conversion factor for converting the product to years (e.g. 1 year/365 days)
Cᵥ^{specific} is a specific heat capacity for the human individual. As used in this specification, the term “about” means within 5%.

To find the specific heat capacity Cᵥ^{specific} a mass of food (ΔM) in kg units is given to an individual and then his or her change in temperature (ΔT) is measured from beginning to end of process. ΔM=Q/(5000 kcal/kg times 4.18 joules/cal) where Q is the heat energy consumption rate. The specific heat cᵥ is the ‘dimensionless’ DoF heat capacity of the individual whose value can be multiplied by 1,197 J/kg·K to get the ‘specific’ heat capacity (c_{V}^{Specific}) in J/kg·K units.

For example,

$${C}_{v} = \frac{D {H}_{S T O R E} ( Heat Stored in Body )}{BODY MASS \times ( {T}_{Initial} - {T}_{Final ( after a few minutes )} )}$$

where DH_{STORE}=DH_{METABOLIC}−(DH_{RADIATION}+DH_{CONVECTION}+DH_{EVAPORATION}); DH_{RADIATION}=0.5×A×(T_{SKIN}−T_{OBJ}); DH_{EVAPORATION}(J/min)=2430 (J/g)×V_{sweat}^{Q}(g/min); DH_{CONVECTION}=0.5×(T_{SHELL}−T_{AIR}) in kJ/min and A is area, V_{sweat}^{Q} is volume. Also see Chapter 21 in Textbook in Medical Physiology and Pathophysiology, 2nd edition, Poul-Erik Paulev MD, Dr.Med.Sci; published by Copenhagen Medical Publishers 1999-2000.

The above equation was derived using a linger-thermo model for a human as show below. Although this equation assumes a 100 kg individual, computer simulations with 50 kg and 70 kg individuals revealed that this mass-independent equation yields the same lifespan results according to:

$$S = kJ ln ( \frac{{e}_{v}^{c} {e}^{n} q ( η )}{{J}^{η}} = \frac{τ}{Δ τ} = \text{...} ) \qquad equation 13$$

where η is a degree of freedom coupling constant that is within 0.79 to 0.82; J is the number of thermote particles (e.g. about 7.24×10³⁸); q(n) is the coupling molecular partition factor (e.g. about 1.088×10⁴); k is the Boltzmann constant; and S is the Boltmnann entropy. The average dimensionless heat capacity cᵥ for a 100 kg individual (with an adult lifespan of 62 years) is about 2.901 while the specific heap capacity c_{V}^{Specific} is about 3470 J/kg·K.

Given equation 13 it follows that:

$$\frac{τ}{Δ τ} = \frac{{e}_{v}^{c} {e}^{n} q ( η )}{{J}^{η}} \qquad equation 14$$

Given the relationship between cᵥ and c_{V}^{Specific} using the heat capacity of liquid water at 310 k (the major component of the human body):

$$\frac{{c}_{ν}}{7 \text{/} 2} = \frac{{c}_{ν}^{s p e c i f i c}}{4186 J \text{/} kgK} \qquad equation 15$$

Further given the relationship between J and cᵥ:

J=Mc²/kTc_{V}=(mc²/kTc_{V})(M/m) equation 16

where c is the speed of light, m is the mass of a molecular of water in kg, T is temperature in kelvin and k is the Boltzmann constant. In further view of linger-thermo theory (where g₀ is about 1, I is the average vibrational frequency of water molecule (about 2×10⁻⁴⁷ kg·m²) υ is the average vibrational frequency of water molecule (about 1.5×10⁹ Hz) and σ is the symmetry number of water molecules (about 2)) then q(η) is as follows:

$$q ( η ) = {q}^{e} {{q}^{t} ( {q}^{r} {q}^{v} )}^{\frac{{c}_{v} - 3 / 2}{2}} \approx {{g}_{0} ( \frac{m k T}{2 {πℏ}^{2}} {V}^{2 / 3} )}^{3 / 2} {( \frac{2 IkT}{{σℏ}^{2}} \frac{kT}{2 πℏυ} )}^{\frac{{c}_{V} - 3 / 2}{2}} = {{g}_{0} ( \frac{m k T}{2 π {ℏ}^{2}} {( \frac{M}{1000} )}^{2 / 3} )}^{3 / 2} {( \frac{2 IkT}{{σℏ}^{2}} \frac{kT}{2 πℏυ} )}^{\frac{{c}_{V} - 3 / 2}{2}} \qquad equation 17$$

The coupling factor between water molecules is then given by:

$$η = α ( M ) \frac{{c}_{V} - {c}_{V , Min}}{{c}_{V , Max} - {c}_{V , Min}} = α ( M ) \frac{{C}_{V} - {C}_{V , Min}}{{C}_{V , Max} - {C}_{V , Min}} = α ( M ) \frac{{C}_{V} - 1794 J \text{/} kgK}{3609.9 J \text{/} kgK - 1794 J \text{/} kgK} \qquad equation 18$$

where α(M) is 0.8724346 for M=100 kg.

Advantageously, the specific heat capacity embodiment only uses the specific heat capacity of the individual and there is no need to obtain the mass of the individual.

Exemplary values for α(M) and c_{v,Max} are show below:

| | M = 50 kg | M = 70 kg | M = 100 kg |
|---|---|---|---|
| α(M) | 0.8715213 | 0.8719574 | 0.8724346 |
| c_{v, Max} | 3.018 | 3.018 | 3.018 |

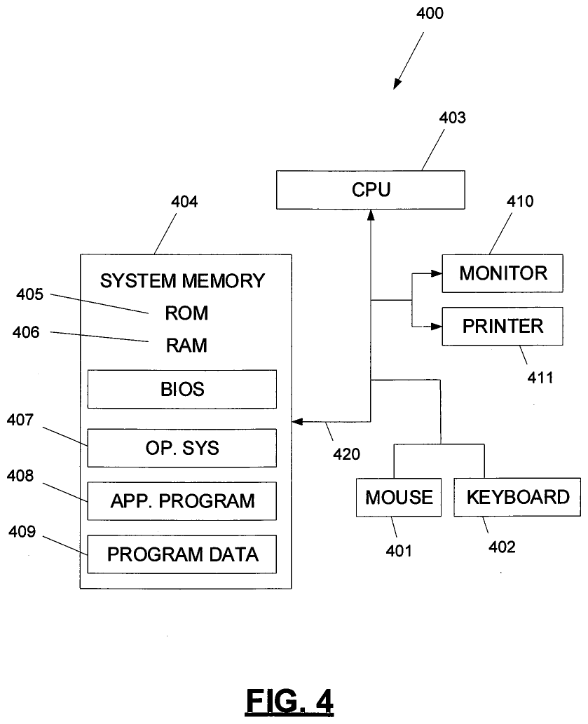

Turning to FIG. 4, there is shown a typical computer system 400 for executing the operations of an application program 408 for carrying out the calculation of life expectancy in accordance with this patent. The computer system 400 has an input apparatus such as a mouse 401 and a keyboard 402 for inputting data and commands to the system 400. System memory 404 includes read only memory (ROM) 405 and random access memory (RAM) 406. RAM 406 holds the BIOS program that allows the system to boot and become operative. RAM 406 holds the operating system 407, the life expectancy application program 408 and the program data 409 in memory 404. Those skilled in the art understand the RAM may be part of the internal memory of the system 400 or may be stored on one or more external memories (e.g. thumb drives, flash RAMs, floppy or external hard disks, not shown) or may be portions of a large internal RAM. A bus 420 carries data and instructions to from system memory 404 to a central processing unit 403. The bus also carries input data user commands form the input mouse 401 and keyboard 402 to the CPU 403 and the system memory 404. Bus 420 also connects the system memory, CPU and input apparatus to output peripherals such as a monitor 410 and a printer 411. In operation, the life expectancy program 408 carries computer readable code to instruct the CPU to carry out the calculation of life expectancy as described above and display the result on the monitor or the printer.

### Calculation of the Nutritional Consumption Rate (ΔM)

In some embodiments, the nutritional consumption rate (ΔM) is not provided by the individual or a proxy and must be received in another manner. In one embodiment, the nutritional consumption rate (ΔM) is received as the result of a calculation.

### Determination of the Nutritional Consumption Rate (ΔM) by GBMI

In one embodiment, the nutritional consumption rate (ΔM) is calculated based on the individual person's GBMI (β_{indiv}) as a function of an appropriately selected optimum GBMI (βₒₚₜ). The value of β_{indiv} is determined using the mass (M) and height (h) of the individual person according to:

$${β}_{i n d i v} = \frac{M}{{h}^{c}} \qquad equation 19$$

An optimum GBMI (βₒₚₜ) is established based on, for example, ethnicity, geographic region (e.g. United States, Japan, etc.) or based on the muscularity/body frame. For example, for the United States, a GBMI (βₒₚₜ) may be set to 25. By way of further example, for Japan, a GBMI (βₒₚₜ) may be set to 23. In one embodiment, the nutritional consumption rate (ΔM) is calculated from the GBMI (βₒₚₜ) according to:

$$Δ M = \frac{{β}_{o p t} + k ( x ) \vert {β}_{i n d i v} - {β}_{o p t} \vert}{{β}_{o p t}} \sqrt{\frac{Δτ}{{τ}_{max}}} M \qquad equation 20$$

where k(x) is a positive number that is a function of the demographic parameter vector x with the value of k(x) determined using actuarial tables.

In another embodiment, the body volume index (BVI) is used instead of the body mass index.

### Determination of the Nutritional Consumption Rate (ΔM) by WHR

In one embodiment, the nutritional consumption rate (ΔM) is calculated based on the individual person's waist-to-hip ratio (WHR, γ_{indiv}) as a function of an appropriately selected optimum WHR, (γₒₚₜ). The value of γ_{indiv} is determined using the waist measurement (w) and hip (H) of the individual person according to:

$${γ}_{indiv} = \frac{w}{H} \qquad equation 21$$

An optimum WHR (γₒₚₜ) is established based on, for example, ethnicity, geographic region (e.g. United States, Japan, etc.) or other demographic information. For example, for the United States, a WHR (γₒₚₜ) may be set to 0.7 for females and 0.9 for males. By way of further example, for Japan, a WHR (γₒₚₜ) may be set to 0.6 for females and 0.8 for males. In one embodiment, the nutritional consumption rate (ΔM) is calculated from the WHR (γₒₚₜ) according to:

$$Δ M = \frac{{γ}_{o p t} + b ( x ) \vert {γ}_{indiv} - {γ}_{opt} \vert}{{γ}_{opt}} \sqrt{\frac{Δ τ}{{τ}_{max}}} M \qquad equation 22$$

where b(x) is a positive number that is a function of the demographic parameter vector x with the value of b(x) determined using actuarial tables.

The methods of determining the nutritional consumption rate (ΔM) described above are only examples. Other suitable methods of determining a nutritional consumption rate (ΔM) would be apparent to those skilled in the art after benefiting from reading this specification. In certain embodiments, a given value of ΔM may be received that leads to clearly erroneous results. For example, a ΔM may be received that results in a theoretical adult lifespan (τ_{theory_adult}) that is greater than the maximum adult lifespan (τ_{Max}). Similarly, a ΔM may be calculated which may result in a theoretical adult lifespan (τ_{theory_adult}) that is greater than the maximum adult lifespan (τₘₐₓ). The method may further comprise the step of verifying the integrity of the calculations by checking against a threshold value (e.g. the maximum adult lifespan (τ_{Max})) and taking corrective action. Examples of corrective action include notifying the user of the error and/or requesting a corrected value of ΔM be supplied.

In view of the foregoing, embodiments of the invention include the ratio of the individual's mass to the individual's nutritional consumption rate when predicting individual lifespan. A technical effect is to permit more accurately predictions for the lifespan of an individual.

### Time Compression

Since an adult individual of age (A) over eighteen years experiences each day of his life to be shorter than when he first became an adult at age eighteen, the adult presently views X days of life expectancy to be shorter than when the adult viewed these same X days as an eighteen year old. The actual amount of this time compression has been found using a linger-thermo model for a human. More specifically, this time compression factor (CF_{A}) is given by:

$$C {F}_{A} = \frac{{τ}_{childhood} + {Δτ ( \frac{{M}_{A}}{Δ {M}_{A}} )}^{2} - A}{{Δτ ( \frac{{M}_{c h i l d h o o d}}{Δ {M}_{childhood}} )}^{2}} \qquad equation 23$$

In the equations above, Δτ is a conversion factor (e.g. 1 year=365 days), M_{A} is the mass of the individual as an adult, M_{childhood} is the mass of the individual at the end of childhood (e.g. τ_{childhood}=18 years, ΔM_{A} is the nutritional consumption rate, ΔM_{childhood} is a nutritional consumption rate for a new adult (e.g. eighteen year old). In one embodiment, ΔM_{childhood} is determined by solving the equation below for an individual of a given mass.

$$Δ {M}_{c h i l a h o o d} = \frac{{M}_{childhood}}{\sqrt{\frac{{Γ}_{max} - {τ}_{childhood}}{Δ τ}}} \qquad equation 24$$

It should be appreciated that the childhood lifespan (τ_{childhood}) is often set to be 18 years, as is traditional in U.S. culture. In other cultures, other values of τ_{childhood} may be used.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

### Example 1: Nutritional Consumption Rate

A system for determining life expectancy is established that sets the childhood lifespan (τ_{childhood}) to eighteen years, the maximum total lifespan (Γₘₐₓ) to 120 years. The parameters of an individual person are received as follows: M=70 kg; age (A)=40 years; ΔM=0.4 kg per day (based on 2000 kcal per day at 5000 kcal per kg); life-expectancy condition=1 (ideal), height=1.6 m; waist circumference 70 cm; hip circumference 100 cm, gender=female; country=US; diet=1 (excellent); ethnicity=1 (Hispanic); fitness=1 (excellent); economic class=1 (middle class); BVI=0 (denoting data not available); value of c in GBMI calculation=2. When the aforementioned parameters are received, steps 202, 204 and 206 have been performed. The individual's theoretical adult lifespan is then determined as follows:

$${τ}_{t h e o r {γ}_{-} adult} = Δ {τ ( \frac{M}{Δ M} )}^{2} = \frac{1 year}{365 days} {( \frac{70 kg}{0.4 kg per day} )}^{2} = 84 years \qquad equation 25$$

Using the set value of eighteen for the childhood lifespan (τ_{childhood}), a theoretical total lifespan (Γ) is determined according to:

Γ=τ_{theory_adult}+τ_{childhood}=84 years+18 years=102 years equation 26

Actuarial tables are consulted and a suitable probability of survival (p_{A}) is chosen based on the individual person's demographic data. In the hypothetical example 1, p_{A} is 0.95 and the current age (A) is 40 years. An expected lifespan (F) is determined as follows:

F=p_{A}(Γ−A)=0.95(102 years−40 years)=59 years equation 27

### Example 2: Nutritional Consumption Rate

A system for determining a life expectancy is established that is substantially identical to example 1 except in that the ΔM is determined to be 0.52 kg per day (based on 2600 kcal per day at 5000 kcal per kg). The individual's theoretical adult lifespan is then determined as follows:

$${τ}_{t h e o r {y}_{-} a d u l t} = Δ {τ ( \frac{M}{Δ M} )}^{2} = \frac{1 year}{365 days} {( \frac{70 kg}{0.52 kg per day} )}^{2} = 50 years \qquad equation 28$$

Using the set value of eighteen for the childhood lifespan (τ_{childhood}), a theoretical total lifespan (Γ) is determined:

Γ=τ_{theory_adult}+τ_{childhood}=50 years+18 years=68 years equation 29

Actuarial tables are consulted and a suitable probability of survival (p_{A}) is chosen based on the individual person's demographic data. In the hypothetical example 1, p_{A} is 0.95 and the current age (A) is 40 years. An expected lifespan (F) is determined as follows:

F=p_{A}(Γ−A)=0.95(68 years−40 years)=27 years equation 30

By contrasting examples 1 and 2 it is apparent the individual in example 2 has a reduced expected lifespan (F) as a result of the increased consumption. It is important to recognize this reduced expected lifespan (F) is not the result of obesity (the example presumes a constant mass of 70 kg for both individuals) but is believed to be the result of metabolic strain experienced by burning more calories per day in order to maintain the 70 kg weight.

### Example 3: Nutritional Consumption Rate

A system for determining a life expectancy is established that is substantially identical to example 2 except in that the mass (M) of the individual is 91 kg. The nutritional consumption rate remains 0.52 kg per day (based on 2600 kcal per day at 5000 kcal per kg). The individual's theoretical adult lifespan is then determined as follows:

$${τ}_{t h e o r {y}_{-} a dult} = Δ {τ ( \frac{M}{Δ M} )}^{2} = \frac{1 year}{365 days} {( \frac{91 kg}{0.52 kg per day} )}^{2} = 84 years \qquad equation 31$$

Using the set value of eighteen for the childhood lifespan (τ_{childhood}), a theoretical total lifespan (Γ) is determined:

Γ=_{theory_adult}+τ_{childhood}=84 years+18 years=102 years equation 32

Actuarial tables are consulted and a suitable probability of survival (p_{A}) is chosen based on the individual person's demographic data. In the hypothetical example 1, p_{A} is 0.95 and the current age (A) is 40 years. An expected lifespan (F) is determined as follows:

F=p_{A}(Γ−A)=0.95(102 years−40 years)=59 years equation 33

By contrasting examples 1 and 3 it is apparent both individuals have the same expected lifespan (F) despite the individual of example 3 being heavier and consuming more energy.

### Example 4: Nutritional Consumption Rate

A system for determining a life expectancy is established that is substantially identical to example 1 except in that the ΔM for the individual person is not known or is not provided. The ΔM is calculated based on the GBMI of the individual. An individual GBMI (δ_{indiv}) is calculated using the mass (M) and height (h) of the individual person as follows:

$${β}_{i n d i v} = \frac{M}{{h}^{c}} = \frac{70}{{1.6}^{2}} = 27.3437 \qquad equation 34$$

Based on demographic information, an optimum GBMI (βₒₚₜ) is set at 25. A value of 0.947 is set for k(x) based on the demographic profile of the individual. The value of ΔM is then calculated as shown below:

$$\begin{matrix} Δ M = \frac{{β}_{o p t} + k ( x ) \vert {β}_{i n d i v} - {β}_{o p t} \vert}{{β}_{o p t}} \sqrt{\frac{Δ τ}{{τ}_{max}}} M & equation 35 \\ Δ M = \frac{25 + 0 .947 \vert 27.3437 - 25 \vert}{25} \sqrt{\frac{1 / 356}{102}} 70 = 0.4 \; 000 kg per day & equation 36\end{matrix}$$

The individual's theoretical adult lifespan is then determined as follows:

$${τ}_{t h e o r {y}_{-} a d u l t} = Δ {τ ( \frac{M}{Δ M} )}^{2} = \frac{1 year}{365 days} {( \frac{70 kg}{0.40 kg per day} )}^{2} = 84 years \qquad equation 37$$

Using the set value of eighteen for the childhood lifespan (τ_{childhood}), a theoretical total lifespan (Γ) is determined:

Γ=_{theory_adult}+τ_{childhood}=84 years+18 years=102 years equation 38

Actuarial tables are consulted and a suitable probability of survival (p_{A}) is chosen based on the individual person's demographic data. In the hypothetical example 1, p_{A} is 0.95 and the current age (A) is 40 years. An expected lifespan (F) is determined as follows:

F=p_{A}(Γ−A)=0.95(102 years−40 years)=59 years equation 39

By contrasting examples 1 and 4 it is apparent both individuals have similar expected lifespan (F) despite the calculation of example 4 not having access to the nutritional consumption rate of the individual.

### Example 5: Nutritional Consumption Rate

A system for setting a life expectancy is described for a 48 year-old person (A=48) with a mass of 70 kg (M=70 kg). This individual was determined to have a nutritional consumption rate of 0.405 kg of food per day (ΔM_{A}=0.405 kg per day). An idealized ΔM_{childhood} of 0.363 is calculated (120 years−18 years=102, M=70 kg). In this example, the mass of the individual at age 18 and at age 48 are both 70 kg.

$$C {F}_{A} = \frac{{τ}_{childhood} + Δ {τ ( \frac{{M}_{A}}{{ΔM}_{A}} )}^{2} - A}{{Δτ ( \frac{{M}_{c h i l d h o o d}}{Δ {M}_{childhood}} )}^{2}} = \frac{18 + \frac{1 year}{365 days} {( \frac{70 kg}{0.405 kg per day} )}^{2} - 48}{\frac{1 year}{365 days} {( \frac{70 kg}{0.3 \; 63 kg per day} )}^{2}} = 0.51 \qquad equation 40$$

Based on this CF_{A} value, a new premium rate can be determined. In the example, a current premium P_{Current} ($100) is multiplied by the compression factor CF_{A} and a function ƒ which, in the example is multiplying by a factor of 1.86.

P_{New}=ƒ(CF_{A})×P_{Current}=1.86(0.51)×$100=$95 equation 41

### Example 6: Specific Heat

A life expectancy calculation is described for an individual with a specific heat Cᵥ^{Specific} of 3456.5 J/kgK.

$${τ}_{t h e o r {y}_{adult}} = Δτ \cdot 3.515 \times {10}^{31} {( 4.872 \times 1 {0}^{- 38} \cdot 3456.5 )}^{0.00048042 ( 3456.5 - 1794 )} = 102 years \qquad Equation 42$$

Advantageously, this permits the calculation of a predicted adult lifespan that is mass independent.

### Example 7: Specific Heat

A life expectancy calculation is described for an individual with a specific heat Cᵥ^{Specific} of 3462.4 J/kgK.

$${τ}_{t h e o r {y}_{a d u l t}} = Δτ \cdot 3.515 \times {10}^{31} {( 4.872 \times 1 {0}^{- 38} \cdot 3462.4 )}^{0.00048042 ( 3462.4 - 1794 )} = 82 years \qquad Equation 43$$

Examples 6 and 7 clearly show predicted lifespan that are different for two individuals with different specific heats and that these different lifespans are independent of the individual's mass.

### Example 8: Specific Heat

A life expectancy calculation is described for an individual with a specific heat Cᵥ^{Specific} of 3470 J/kgK.

$${τ}_{t h e o r {y}_{a d u l t}} = Δτ \cdot 3.515 \times {10}^{31} {( 4.872 \times 1 {0}^{- 38} \cdot 3470 )}^{0.00048042 ( 3470 - 1794 )} = 62 years \qquad Equation 44$$

Examples 6 and 8 clearly show predicted lifespans that are different for two individuals with different specific heats and that these different lifespans are independent of the individual's mass.

### Example 9: Specific Heat

### Equation 45:

A life expectancy calculation is described for an individual with a specific heat Cᵥ^{Specific} of 3480.5 J/kgK.

$${τ}_{{theory}_{adult}} = Δτ \cdot 3.515 \times {10}^{31} {( 4.872 \times 1 {0}^{- 38} \cdot 3480.5 )}^{0.00048042 ( 3480.5 - 1794 )} = 42 years \qquad$$

Examples 6 and 9 clearly show predicted lifespans that are different for two individuals with different specific heats and that these different lifespans are independent of the individual's mass. Example 9 specifically illustrates a dramatic shorting of lifespan that can occur under strained metabolic conditions.

What is claimed is:

1. A method for maintaining a healthy mass, the method comprising steps of:
measuring a mass (M) of an individual person;
calculating a nutritional consumption rate (ΔM) according to:
$$Δ M = M \sqrt{\frac{Δ τ}{{τ}_{t h e o r {y}_{-} a d u l t}}}$$
wherein Δτ is a conversion factor for converting the nutritional consumption rate to calories per day and τ_{theory_adult} is a targeted adult lifespan;
consuming, by the individual person, a daily amount of calories that is within 1% of the nutritional consumption rate (ΔM);
performing, by the individual person, a daily amount of exercise to expend a total amount of calories per day within 1% of the nutritional consumption rate (ΔM).

2. The method as recited in claim 1, further comprising a step of determining a basal metabolic rate (BMR) for the individual person.

3. The method as recited in claim 2, wherein the total amount of calories per day includes the basal metabolic rate (BMR).

4. A method for maintaining a healthy mass, the method comprising steps of:
measuring a mass (M) of an individual person;
calculating a nutritional consumption rate (ΔM) according to:
$$Δ M = M \sqrt{\frac{Δ τ}{{τ}_{t h e o r {y}_{-} a d u l t}}}$$
wherein Δτ is a conversion factor for converting the nutritional consumption rate to calories per day and τ_{theory_adult} is a targeted adult lifespan;
consuming, by the individual person, a daily amount of calories that is within 1% of the nutritional consumption rate (ΔM);
performing, by the individual person, a daily amount of exercise to expend a total amount of calories per day within 1% of the nutritional consumption rate (ΔM);
measuring a second mass (M) of the individual person after the steps of consuming and performing have been repeated for at least one week;
if the second mass is equal to the mass (M), repeating the steps of consuming and performing;
if the second mass is greater than the mass (M):
reducing the daily amount of calories by 5% to produce a reduced daily calorie amount, while maintaining the daily amount of exercise;
consuming, by the individual person, the reduced daily calorie amount;
performing, by the individual person, the daily amount of exercise;
if the second mass is less than the mass (M):
reducing the daily amount of exercise by 5% to produce a reduced daily exercise amount, while maintaining the daily amount of calories;
consuming, by the individual person, the daily amount of calories;
performing, by the individual person, the reduced daily amount of exercise.

5. The method as recited in claim 4, further comprising a step of determining a basal metabolic rate (BMR) for the individual person.

6. The method as recited in claim 5, wherein the total amount of calories per day includes the basal metabolic rate (BMR).
